(19) United States
(12) Patent Application Publication
(10) Pub. No.: US 2015/0149212 A1
(43) Pub. Date: May 28, 2015

(54) PATIENT INFORMATION INTERFACE

(71) Applicants: Jerome Rolia, Kanata (CA); Sujoy Basu, Sunnyvale, CA (US); Sharad Singhal, Belmont, CA (US); Akhil Kumar, State College, PA (US); Wen Yao, State College, PA (US)

(72) Inventors: Jerome Rolia, Kanata (CA); Sujoy Basu, Sunnyvale, CA (US); Sharad Singhal, Belmont, CA (US); Akhil Kumar, State College, PA (US); Wen Yao, State College, PA (US)

(21) Appl. No.: 14/397,636

(22) PCT Filed: Jun. 8, 2012
(86) PCT No.: PCT/US2012/041572

§ 371 Date: Oct. 28, 2014

(51) Int. Cl. G06F 19/00

(52) U.S. Cl. CPC G06F 19/345; G06F 19/322; G06F 19/325
USPC 705/3

(57) ABSTRACT

Examples of the present disclosure may include methods, systems, and computer readable media with executable instructions. An example method for providing a patient information interface can include accessing patient health record (PHR) information for a particular patient, and referencing medical care guideline information. A medical process associated with the particular patient is determined based on the PHR information and medical care guideline information, and context information associated with the particular patient and/or a healthcare system is received in a medical process-aware manner to a context model implemented on a computing system, including clinical, logistical and operational information. Context aware information is presented, via an output device of the computing system based on the determined medical process and context information.

### BACKGROUND

Rapidly growing patient interests in participating in their care process and accessing their health data has motivated health organizations to provide patient-oriented care delivery both in clinical and homecare settings, with the goal of giving patients a more proactive role in their own care. Despite the advances in life expectancy and quality of life, the current healthcare delivery system faces significant challenges in terms of cost, accessibility and quality.

### BRIEF DESCRIPTION OF THE DRAWINGS

FIG. 1 illustrates an example medical care clinical workflow in a healthcare system.

FIG. 2 illustrates a block diagram of an example computing system for implementing a Patient Information Interface (PII) in accordance with one or more examples of the present disclosure.

FIG. 3 illustrates a decision tree of a partial patient conversation model in accordance with one or more examples of the present disclosure.

FIG. 4 illustrates a flow diagram of operation for a workflow engine portion of a computing system for implementing a PII in accordance with one or more examples of the present disclosure.

FIG. 5 illustrates a block diagram of an example machine readable medium in communication with processing resources in accordance with one or more examples of the present disclosure.

FIG. 6 illustrates a method for PII in accordance with one or more examples of the present disclosure.

### DETAILED DESCRIPTION

Patient-centric healthcare delivery can provide medical care that is respectful of, and responsive to, individual patient preferences, needs and values. Patient values can be used to guide clinical judgments and decisions. As mobile devices become pervasive and access to health information becomes easier so that patients can become more informed, patients can participate more in decision making about their health matters. The various embodiments of the Patent Information Interface (PII) of the present disclosure provide a way to foster better patient-centric care service delivery by providing context aware information to the patient based on integrated consideration of patient health records, medical care guidelines, and/or context information associated with a healthcare system.

A formal process-driven framework can support stream-lined patient communication. The framework can give patients access to more information to interact with medical care providers and take a more active role in their own medical care. Their preferences and values can be sought and integrated in medical decision making. The framework also can keep track of patient and clinical decisions, actions, and outcomes at various points along a medical care episode and/or medical process, for example, so that information that can impact medical care can be efficiently made available to discrete medical care providers.

As used herein, medical process refers to a medical objective, and medical care episode refers to an instance of medical care within the medical process. For example, a medical process may be heart surgery to correct a heart-related defect. In the course of the heart surgery process, a patient may experience several medical care episodes in preparation for surgery, undergoing the actual surgery and recovery therefrom. For example, the various discrete medical care episodes that a patient may be subjected to related to the heart surgery medical process can include a consultation with a general practitioner to evaluate the patient's ability to tolerate surgery, a consultation with a cardiologist, a consultation with the heart surgeon, a CAT scan, MRI, sonogram, angiogram, and/or other discrete tests to obtain medical information, the surgery itself and associated hospital stay, and/or further consultations with surgeon, cardiologist, general practitioner, dietician, physical therapist, and/or financial/insurance specialists, etc.

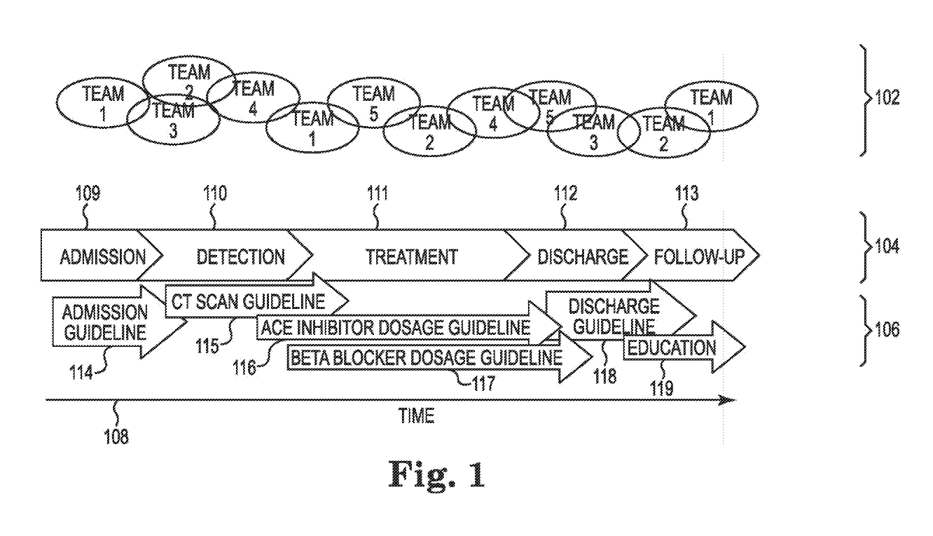

FIG. 1 illustrates an example medical care clinical workflow 104 in a healthcare system. A clinical workflow 104 can correspond to a medical care process, e.g., heart surgery, and/or a particular medical care episode within a larger medical care process, e.g., pre-op CT scan in preparation for heart surgery. A clinical workflow 104 can delineate a path for a patient through the various steps in interacting with medical care providers, e.g., clinic, lab, pharmacy, administration, insurance, social work, and other participants, in the healthcare system over time 108. The clinical workflow 104 shown in FIG. 1 might involve, for example, a medical care facility, e.g., hospital, clinic, emergency care practice, visit that include admission 109, detection 110, treatment 111, discharge 112, and follow-up 113 portions.

Various guidelines 106 can be associated with one or more portions of the clinical workflow 104. For example, the patient may be processed through admission 109 to the medical care facility based on an admission guideline 114, which might be administrative in nature and particular to a facility. Alternately, the admission guidelines 114 might also include health insurance requirements and procedures for medical care facility admission, which might be particular to certain insurance policy holders and might depend on agreements between the hospital and an insurance company, among other considerations.

A clinical workflow 104 can correspond to a medical process, which may involve a plurality of medical care episodes, or can correspond to a single medical care episode. A medical care episode can begin, for example, with admission to a medical care facility, e.g., a hospital, clinic, and can be complete upon discharge and/or follow-up care. The patient may interact with multiple teams of medical care providers, e.g., doctors, nurses, and lab technicians at the medical care facility. As used herein, medical care providers can also include healthcare and insurance administrative personnel (to the extent they are integral to providing and/or payment of medical care services). Further, medical care, as used herein, can include dental services, as well as other type of health care.

The patient may then continue to receive follow-up services from social care agencies that may also be part of a healthcare system. The care that is provided may be according to one or more guidelines or best practices, such as medical, administrative, insurance guidelines, among others. In general, a care episode may span multiple care providers. Therefore, the Personal Health Record (PHR) can be a good place to maintain a summary of the patient's care so that relevant information can be shared by authorized medical care providers in different administrative domains, e.g., different healthcare organizations.

Various embodiments of the present disclosure utilize the PHR along with context information. A workflow engine implements process management mechanisms, thereby providing location aware services, process recording, and a rule-based decision tree. Location aware services can rely on global positioning and other locating services to determine location of a patient and medical care providers. A context model captures context information such as context variables, e.g., patient location, patient mobility, etc., and possible relationships involving the context information, e.g., between context variables, between context information and information contained in the PHR, etc. At run time, rules can be triggered by context information, which can generate actions that map to medical care services customized to needs of a particular patient.

Context generally refers generally to an environment, e.g., a whole situation or background relevant to a particular circumstance such as a medical care episode, event, decision, condition, etc. With respect to a medical patient, depending on previously-obtained information regarding a patient, such as by an answer to a previously-asked question for instance, a subsequent question can be adjusted or customized based on the context. For example, a negative answer to a previous question regarding any allergies might be used to modify a subsequent question regarding a particular allergy that may be triggered by a specific drug. A contextual model with clinical, logistical, and operational information is used to help improve the patient experience in the manner described herein. Context information provides opportunities to enhance the patient's interaction with the health system both clinically and logistically. Context information includes clinically related information including the type of process the patient is involved in, identification of provider(s) they are receiving care from, providers that are available to them, their preferences are in terms of care, etc. Logistical context information includes a patient's current or expected physical location, directions to the location where a patient must go, and instructions on how to receive a service. Operational context can be the relationship between guidelines, e.g., medical, administrative, procedural, social care, and may be influenced by policy for a healthcare system. In general, a process is an enactment of one or more medical, administrative, and/or social care guidelines.

Medical guidelines are best practices for care that are typically established using evidence based methods and/or are based on regulatory requirements. Guidelines are published nationally and are typically adapted for use in particular healthcare settings. Medical guidelines have been modeled as decision trees that guide the care of a patient. Each decision node in the tree corresponds to a question and branches based on possible answers. The choice of branch is determined by the answer.

A medical guideline, such as a clinical protocol or clinical practice guideline is a document that guides decisions and criteria regarding diagnosis, management, and treatment, in specific areas of healthcare. For example, guidelines can be categorized by disease types like pediatrics, pulmonary or heart diseases. This is naturally aligned with the way they are developed, e.g., by medical staff with different expertise areas.

By including context information related to such guidelines and the operational and logistical relationships between them and the medical care providers that act according to them in the approach of the present disclosure, situational awareness is enabled for the healthcare system. Embodiments of the present disclosure use interactions with a patient to obtain and provide information related to guidelines to improve patient experience. The information may be related to likely outcomes that a patient should be made aware of based on current knowledge or may be related to problems or issues that are typical for patients in a similar situation.

For example, a patient's current context may cause the context model to initiate an interaction that asks the patient questions that correspond to a guideline that is expected to be applied to the patient. The information can be used to inform the patient of treatment options/outcomes that may be considered by the patient's medical care provider. The interactions may also report to the patient which option is best aligned with the patient's preferences, and why; and which option is most likely to be recommended by the doctor, and why. The information gathered and reported can also be presented to the doctor in a systematic and timely manner so that the doctor is reminded of the options recommended by the guideline(s).

Moreover, one key purpose is to inform the patient and doctor prior to the doctor visit to enable an efficient patient-provider dialog during the visit. The agreed upon decision can be recorded in the PHR. The patient and care provider can provide feedback to the PII that indicates how well the context model prepared the patient and provider so that the context model can be continuously improved. Patients in similar situations may have a common set of issues for which information can be provided that help patients in the similar situations overcome the issues more independently.

Guidelines, e.g., medical guidelines, may suggest certain issues that may affect patient experience. The number of issues may be reduced based on information in a patient's PHR. The issues can be associated with interactions that are presented to the patient to solicit and/or provide additional information to the patient that improves patient experience. These issues can address hand-off challenges that are present when a care process spans multiple administrative domains.

Patients can also face many logistical hurdles. They are often unfamiliar with provider locations, the physical layouts of buildings, and of the protocols for receiving services. As a patient participates in a medical process, the PII can become aware of the patient's location using global positioning system (GPS) technology, or other location and/or identification technology such as radio frequency identification (RFID). In a context specific manner, where context includes location, the PII can make several interactions known to the patient. These interactions may answer questions that are common for patients in that context. An answer may include verbal or textual directions to a location to receive a service, to the nearest restroom, to an open cafeteria, or where to find a particular bus stop or taxi stand, etc.

While such interactions need not be stored in a patient's PHR, it may still be recorded by the PII to enable learning about what is helpful to patients in particular contexts to support continuous improvement of the PII. Other interactions may help to explain a provider's internal protocols for receiving a service. For example, in some facilities it is necessary to take a number before being served. In some facilities it may be necessary to fill in a form before being served. If the information needed for the form is already in the patients PHR then the PII can pre-generate the completed form for review and submission.

Context information relative to a patient can be input to a workflow engine. The workflow engine can process, e.g., consume, consider, evaluate, context information, and determine rule(s) to trigger and PII interactions that need to be invoked to improve patient experience. The PII interactions may obtain or provide information to the patient, using information from the patient's PHR, databases, the Internet, and/or other sources.

The attributes of PEI, including the context information and workflow engine-implemented context model, can be maintained over time to accurately reflect opportunities to improve patient experience within a healthcare system. One notable aspect of the approach presented in this disclosure is that the context model is medical process-aware and can keep track of the exact time sequence of activities and interventions that are performed as a medical care process proceeds. The context model can operate in a medical process-aware manner by customizing patient and/or medical care provider interactions for a specific medical process. That is, the context model can solicit context information that is relevant to a specific medical process. Context information that is relevant to a specific medical process includes the values for context variables that can cause a modification to the medical process. For example, a medical process may preferably involve treatment with a drug that can cause an allergic reaction in some people. As such, the context model can cause a patient interaction to solicit whether the patient has the allergy of interest to the specific medical process (and to not query the patient regarding the allergy if a medical process does not involve treatment with the drug that can cause the allergic reaction.) Further, the context model is knowledge-dependent since the interactions for different medical processes, e.g., heart failure, pediatrics, might be significantly different.

Referring again to FIG. 1, the patient may receive a CT scan according to a CT scan guideline 115 during the detection 110 portion of the clinical workflow 104. Results of the CT scan might suggest a heart condition, for which the patient may be prescribed appropriate drugs, e.g., an ACE inhibitor and beta blocker, during the treatment 111 portion of the clinical workflow 104 according to an ACE inhibitor dosage guideline 116 and/or a beta blocker dosage guideline 117. The patient may then be discharged 112 from the hospital based on a discharge guideline 118, and may further receive outpatient care during the follow-up 113 portion of the clinical workflow 104. The follow-up 113 can include education 119 appropriate to the detected medical condition.

While FIG. 1 shows a simplified clinical workflow 104 for an example medical care process. The nature and arrangement of the portions of the clinical workflow 104 are not limited to those shown in example by FIG. 1. For example, detection 110 may occur prior to admission to a medical care facility for treatment. Detection 110 and treatment 111 portions of a clinical workflow 104 can involve more and different activities subject to other guidelines 106 than those shown in FIG. 1.

FIG. 1 further shows that a plurality of distinct groups of medical care providers 102, e.g., teams 1-5, can be involved in the clinical workflow 104. The medical care providers can include administrative personnel, nurses, doctors, technicians, and others. Distinct groups of medical care providers 102 can be in different departments, at different locations, on different shifts, etc. Two teams can work together at times, such as team 2 and team 3 during detection 110, or may work sequentially, such as care of the patient being passed from team 4 in detection 110 to team 1 for treatment 111.

For example, when a patient schedules an appointment, or is discharged from a medical care facility, the patient may communicate with administrative staff. At other points in the clinical workflow 104 the patient may undergo clinical activities such as detection 110 and treatment 111, which can involve people from various medical departments/specialties, staff, resources, etc. In a medical care process and/or a care episode within the medical care process, the patient might be the only entity involved throughout the clinical workflow 104. As such, a process perspective, such as is illustrated by the clinical workflow 104 shown in FIG. 1, can be used to maintain coordination and flow of information between the patient and medical care providers 102 to in support of an optimal medical care outcome.

Due to limited resources and/or lack of emphasis on patient-centric care, a healthcare system, e.g., organization(s), can be operated inefficiently from a patient perspective, offer poor quality of service to patients, and/or provide limited resources for patients to access their health care data in order to participate in medical decision making. Cost, accessibility, and quality of service delivery are largely a function of the operation of a healthcare system. Other challenges, such as longer life spans are resulting in an aging population having greater incidence of chronic illnesses and increases in overall healthcare spending, and/or limited medical care provider time and limited access of a patient to their own health information can lead to a patient being unaware of the medical processes to which the patient is undergoing, the duration, location, medical provider, and cost associated with respective portions of the clinical workflow 104.

As a result, patients can get confused and/or frustrated when they do not know what to expect at various points of care. Furthermore, patients may not understand the advantages, disadvantages, risks, costs, and detailed scope of procedures that may be associated with each option. Some patients can feel overwhelmed by uncertainty and/or feel disconnected from the clinical workflow 104 as a result of a lack of appropriate and timely information relevant to their medical care. To address the above-mentioned problems, the PII of the present disclosure utilizes a process-driven approach to streamline information flow to the patient and/or medical care providers, thereby facilitating improved patient-centric care.

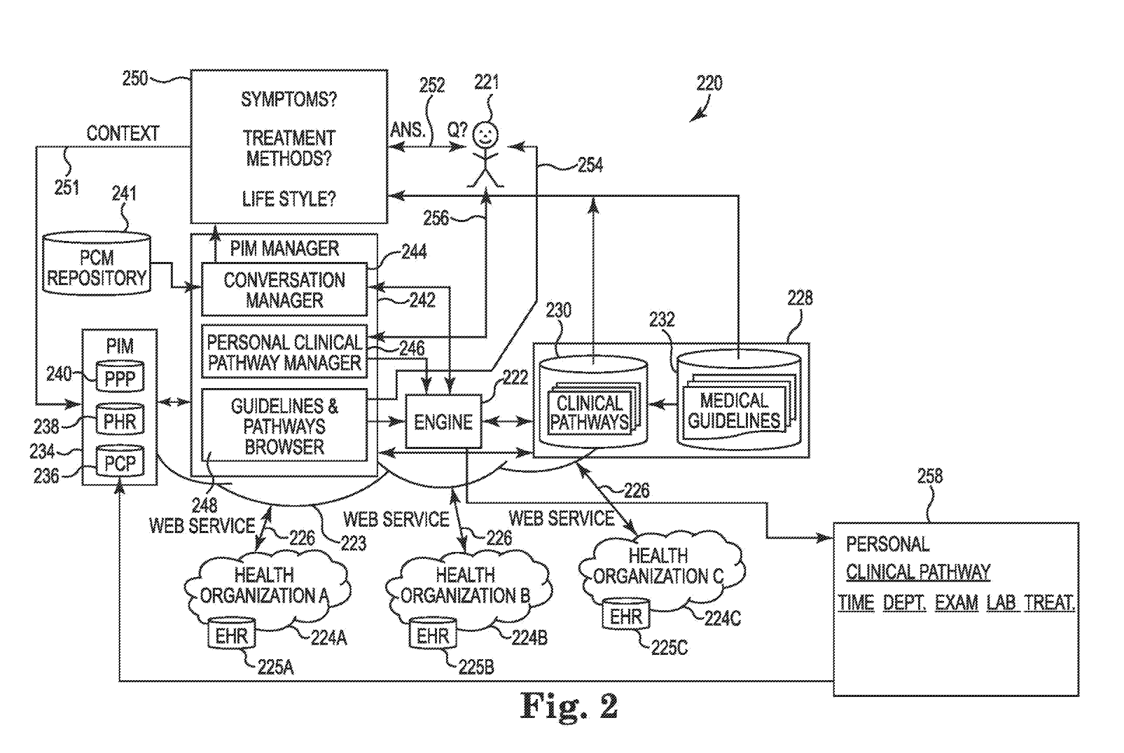

FIG. 2 illustrates a block diagram of an example computing system for implementing a PII 220 in accordance with one or more examples of the present disclosure. FIG. 2 shows one example of a PII 220. According to some embodiments, the computing system used to implement the PII can use a variety of open source tools. According to various embodiments, a computer can be used to implement a PII 220 with formalized clinical workflows based on the medical record of the patient, various guidelines, e.g., medical care guidelines, administrative procedures, and other context information. The computer-implemented PII 220 can incorporate patient needs, resource availability to the patient, and patient preferences as part of the context information. In this manner, a computer-implemented PII workflow engine 222 can determine possible alternative clinical decisions based on medical and healthcare system best practices, as well as patient characteristics and preferences.

The PII 220 can include a Patient Information Model (PIM) 234. The PIM 234 can maintain three parts of patient data, including a Personal Clinical Pathway (PCP) 236, PHR 238, and Personal Preference Profile (PPP) 240. All steps and decisions are guided or driven by medical guidelines and patient preferences, needs, and values can be captured in the PIM 234. A context model is implemented in the PIM 234 based on the contents of the PCP, PHR, and PPP.

A PHR 238 can be a patient's lifelong health information. The patient can access their PHR 238, and the PHR 238 can be used to coordinate medical care and be shared with other entities, such as medical care providers. The PHR 238 might include patient-reported symptoms, electronic health records released by the doctor's office to the patient, prescriptions and lab results all of which have been uploaded by patients, or even data from smart devices, among others.

The PHR 238 can be maintained and/or updated by the patient, medical care providers, and/or healthcare organization, e.g., assuming entity maintaining the PHR 238 is authorized to do so by the patient. The PHR 238 can include data from many healthcare organizations. The PHR 238 can be electronic, and can be made accessible online at any time, for example, being stored in a cloud-based architecture (computing arrangement) 223 accessible by a web service 226. For example, the PHR 238 of a particular patient 221 may be used by healthcare organization A 224A to obtain medical information from the PHR 238 to update the electronic health records (EHR) 225A that healthcare organization A 224A maintains for the particular patient 221. Also, the PHR 238 can also be updated from the EHR 225A by healthcare organization A 224A. Sharing of medical information between the PHR 238 and EHR 224A reflects that a patient can have medical information originating from many different healthcare organizations, which can be assembled into a comprehensive record in the PHR 238.

Similarly, the PHR 238 of the particular patient 221 may also be used by health organization B 224B for exchanging medical information between the PHR 238 and the EHR 225B maintained by health organization B 224AB. Likewise, the PHR 238 of the particular patient 221 may also be used by health organization C 224C for exchanging medical information between the PHR 238 and the EHR 225C maintained by health organization C 224C. Use can include consultation and/or modification, based on access permissions, for example.

In this manner it is possible for medical information regarding a particular patient 221 to flow between health organizations via the PHR 238. For example, a first medical care episode may occur at healthcare organization C 224C, which can be recorded in the EHR 225C corresponding to the particular patient 221. The PHR 238 of the particular patient 221 can be updated from the EHR 225C. Subsequently, a second medical care episode involving the particular patient 221 may occur at healthcare organization A 224A. Healthcare organization A 224A may seek to update EHR 225A from the PHR 238 so that medical care providers at healthcare organization A 224A can be apprised of all relevant medical information for the particular patient 221, which may be relevant to the second medical care episode. The second medical care episode can be documented in EHR 225A, which in turn can be used to update the PHR 238.

For example, the PII 220 can interact with the various health organizations 224 through an HU messaging protocol, which has been developed as a leading standard for data integration in heterogeneous systems today. Electronic communications with the PII can utilize a message-based standard for data exchange among applications when certain trigger events occur, e.g., clinical events.

The patient can gain access to copies of their PHR 238 and/or the EHR 225 as captured by a medical care provider, e.g., doctors, clinics, labs, and hospitals, associated with a health organization 224. A Healthcare Information Exchange (HIE) is an example implementation of a federation of medical care providers that enables such an exchange of information.

HIEs can enable the sharing of EHRs. However, the HIEs do not usually organize such information within the context of the specific medical processes and/or guidelines, or the relationships that implicitly structure the delivery of care. Yet, such structuring provides clinical, logistical, and operational context. According to various embodiments of the PII 220 of the present disclosure, clinical, logistical, and operational context is considered along with the PHR. The PIM, PHR, PCP, and/or PPP can be organized in a patient centric and/or care episode centric manner. The PII 220 can provide reminders and alerts, to the patient and/or medical care providers, as well as employing this information to improve a patient's situational awareness of the healthcare system and how to navigate it with less effort.

Process recording enables a process oriented view of records organized according to care episodes. A patient's sequence of interactions with care providers can cause medical records to flow to the PHR. The records can be annotated so they are associated with the appropriate corresponding process and are recognized as steps in the process. Thus a patient or provider can readily view records that correspond to a particular episode. A medical process, its steps, and the PHR can all provide context information.

The PCP 236 records a clinical pathway, e.g., clinical workflow 104 shown in FIG. 1. The PCP 236 documents decisions, actions, and/or outcomes, among other information, organized in chronological order pertaining to the particular patient 221. One example of a PCP record 258 is shown in FIG. 2 as including chronological information such as time/date, organizational information such as a department taking some action, examination notes, lab results, and treatments performed, among other medical information. The contents of the PCP record 258 are not limited to the specific information categories illustrated in FIG. 2.

A PCP record 258 can be stored in the PCP 236. The PCP 236 can allow deviations from best practice to satisfy a patient's preferences, or other context information. A PCP 236 can be compared with similar examples of clinical workflows 104 as stored in the clinical pathway repository 230 or a template that models an aggregate of similar clinical workflows that may also be stored in the clinical pathway repository 230. The PPP 240 can capture preferences, needs, and values pertaining to a current situation of a particular patient 221.

A PPP 240 can reflect various patient preferences, such as by assignment of preference based on a 1-N scale, where a larger number can indicate a stronger preference. Patient preferences can be considered while applying guidelines, e.g., medical guidelines. In this way, the PII can take into consideration an awareness of patients' preferences of treatment methods, e.g., medication or surgery, quality-of-life aspects, e.g., exercise- or diet-based rehabilitation program, etc. A PPP can be acquired from context-building. The medical conditions of patients can take priority over their preferences whenever there is a conflict, especially where patient safety is at issue. For example, although surgery may be a least preferred treatment method of a particular patient, if she has repeated hospitalization despite aggressive medical therapy; such context can be considered in elevating an appropriateness of surgical treatment.

Clinical pathways can be derived from customization of medical guidelines 232, which can be modeled in a formal workflow modeling language, e.g., BPMN 2.0, and stored in a repository 228. In general, clinical practice should follow evidence-based guidelines to ensure an optimal outcome. According to various embodiments of the present disclosure, a Patient Conversation Model (PCM) can be used to organize possible questions that might be presented to a patient at a specific point of care within a medical process, clinical pathway, clinical workflow, and/or particular care episode. Information related to the PCM can be stored in a PCM repository 241. The PCM can be tailored from guidelines and integrates with practical experience.

When a clinical pathway is initialized for a case involving a particular patient 221, a workflow engine 222, e.g., a jBPM workflow engine, can maintain the status of the running medical process, among other functions. The workflow engine 222 may also coordinate between components of the PII. For example, the workflow engine 222 may accumulate discrete information contained in the EHRs 225 of multiple health organizations 224 into the PHR of a particular patient.

The Pit can facilitate patient collaboration with clinical teams to determine their treatment. Thus, the PII can allow patients to: (1) access their health data and gain insights into the whole process; (2) express choices and take more responsibility; and (3) get a more personalized and coordinated continuum of care, e.g., care supply chain. From the provider's perspective, time consuming but redundant aspects of patient communication workload can be transferred from medical staff to the system. Additionally, patient's choices and a continuum of care can be presented to all medical care teams to improve coordination among them, and track clinical pathways so that process improvement can occur. These features enable medical care providers to focus on their work while operational efficiency is improved.

Furthermore, the clinical pathways of many patients can be studied from repository 228 to improve the efficiency and outcomes of pathways in general, and thereby improve the overall healthcare system. Thus, the PII may use as an example a composite clinical pathway developed from the clinical pathways of many patients and stored in repository 228, rather than a generic medical guideline retrieved from the Internet.

According to various embodiments of the present disclosure, the PII can augment a PHR and/or other Electronic Medical Record (EMR) system with context information and a context model that is used to improve patient experience. Clinical, operational, and logistical context information can enable the PII to invoke interactions that obtain and offer information to the patient. This information can improve situational awareness for the patient to reduce the effort needed by the patient to pass through a care episode and/or medical process, such as by educating the patient on what to expect and enable more efficient dialogs with care providers, informing the patient of what services are available and directing the patient how to receive services. Patients and medical care providers can provide feedback to the PII on the solution so that methodologies and context information can be continuously improved.

The workflow engine 222 can trigger the Conversation Manager 244 at various points of care to control and/or facilitate interaction, e.g., conversation, between the PII 220 and patient 221 based on a PCM. As a result of context-building through patient and/or medical care provider interaction(s), information derived from the interactions can be sent to update PIM 234, including PHR 238 and/or PPP 240, through PIM Manager 242.

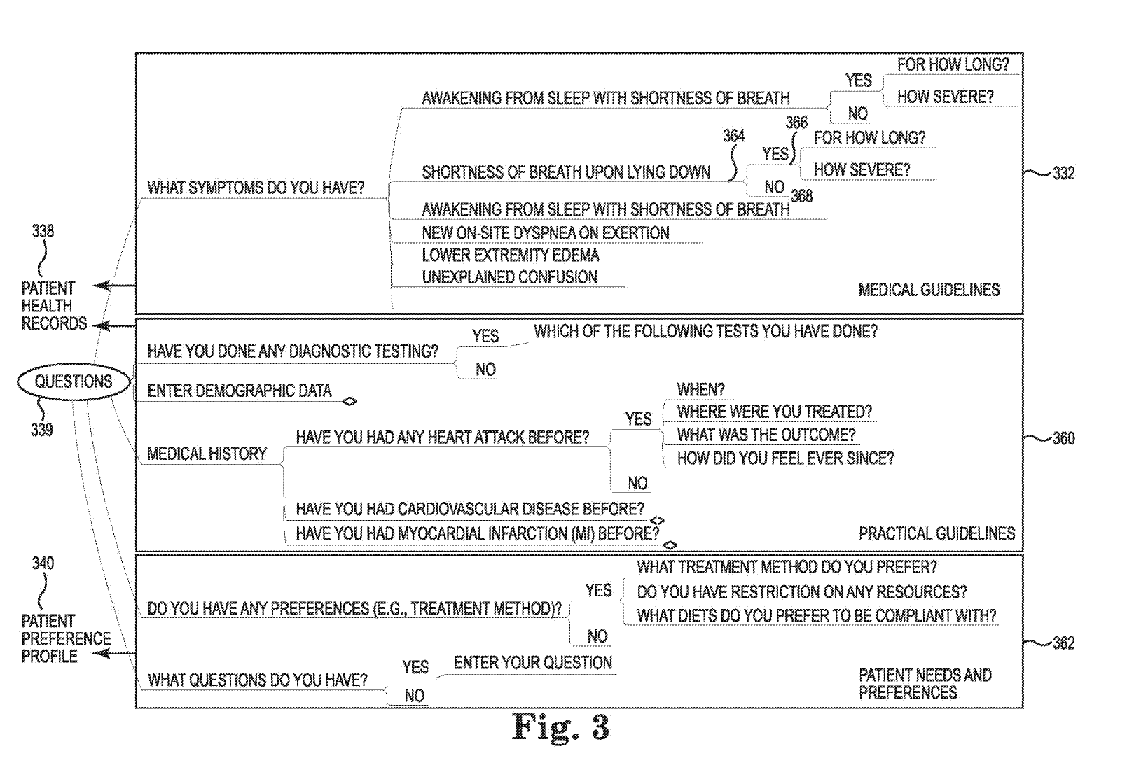

The PIM Manager 242 can include a Conversation Manager 244, a Personal Clinical Pathway Manager (PCP Manager) 246, and a Guidelines and Pathways Browser 248, among other components. The Conversation Manager 244 can execute a conversation model 250 to implement interactions 252 with the patient 221 and/or medical care providers. The interactions 252 can be structured as information and/or questions presented to the patient 221, and information received from the patient 221, such as answers to questions from a decision tree presented to the patient 221. The questions can be related to symptoms, treatment methods, and/or patient life style, among others. The conversation model 250 is discussed further with respect to FIG. 3 below.

The Guidelines and Pathways Browser 248 can implement, for example, Internet search capability, such as might be responsive to a search query. In this manner, the Guidelines and Pathways Browser 248 can provide information to the workflow engine 222 and/or by direct interaction 254 to the patient 221.

The Guidelines and Pathways Browser 248 can provide information in a structured manner consistent with other information presented by the PII that a patient might otherwise have to search for on the Internet in an unstructured manner. For example, an Internet search regarding heart surgery might yield multiple results, which can include conflicting information among the results and/or information that conflicts or is duplicative with other information presented to the patient via the PII. The Guidelines and Pathways Browser 248 can be configured to present medical information to the patient that augments and/or supports the PII, such as from website(s) that have been reviewed for accuracy and/or approved for consistency with other information presented via the PII. Such information can be stored in a repository 228, or more specifically in the Clinical Pathway repository 230 and/or the Medical Guideline repository 232. The Guidelines and Pathways Browser 248 may also retrieve guideline and pathway information directly from the repositories 228, 230, and/or 232.

The Conversation Manager 244, PCP Manager 246, and/or Guidelines and Pathways Browser 248 can each provide information to the workflow engine 222, as necessary. For example, the workflow engine 222 may periodically search the Internet for updates to published medical guidelines 232, and retrieve same for local storage via the workflow engine 222 in a repository 228, e.g., in generic and/or modified form. The guidelines may reflect the actual guidelines that guide and are reported by the medical care providers in the healthcare system as available to the patient.

The PCP Manager 246 can report the clinical pathway of a particular patient to the patient and/or medical care provider by direct interaction. Similarly, the PCP Manager 246 can present historical clinical pathway information for similar patient(s) including a PCP record 258, and/or prospective clinical pathway information such as information regarding suggested and/or scheduled medical care.

As a patient prepares to receive medical care from medical care providers, a new Personal Clinical Pathway 236 is started. The patient can use the Guidelines and Pathways Browser 248 to query the clinical pathways of similar patients and anticipate the guideline(s) that are most likely applicable. A clinical pathway is a historical record, e.g., log of medical care received by a patient. In contrast, a guideline is a prospective, e.g., expected, treatment plan of a medical process to treat a medical condition. The clinical pathway is completed as the patient receives medical care. The clinical pathways of other patients can also be used in a similar fashion as a guideline since a historical record of treatment received by another patient can provide insight into possible expected treatment for a new patient experiencing similar symptoms. However, the clinical pathway of an individual patient may include non-standard medical care based on unique circumstances that may, or may not, be experienced by subsequent patients.

A patient and/or a medical care provider can initiate a clinical pathway. For example, a patient may initiate a clinical pathway for shortness of breath, which can be used to initiate interactions that pre-ask questions of the patient in advance of their meeting with a medical care provider, e.g., regarding symptoms, etc.

Once a starting guideline(s) is selected, or a new guideline is started or anticipated, or any other opportunity arises to pre-ask questions and inform the patient, this causes the engine 222 to direct the PIM Manager 242 to engage with the patient via the Conversation Manager 244. This engagement informs the patient and pre-asks questions whose responses are stored in the PIM 234 as PPP 240 and PHR 238. A question that is motivated by a guideline has its response annotated by the guideline's identifier when stored. Similarly, as the patient receives care from medical care providers according to guidelines, PHR 238 are annotated by guideline identifiers to enable a better understanding of how guidelines relate to a patient context and to one another.

Annotating the PHR and/or questions and other interactions with the patient can indicate the guideline, or series of guidelines, upon which a patient's clinical pathway was following, which can provide context information for informing and/or questioning the patient. Such correlation between PHR, questions and guideline(s) can also facilitate subsequent mining of information. Audits can also be conducted to determine whether the guidelines were applied correctly. Furthermore, where more than one guideline may be of interest, questions can be correlated to the appropriate guideline(s). For example, in moving from one guideline to another, questions can be pre-asked about the next guideline before it is activated, such as by invoking location aware conversations, etc.

The records in the PHR 238 that correspond to a care episode, and may involve a multiplicity of guidelines, are the patient's clinical pathway. When the patient completes the clinical pathway, e.g., after follow-up 113 is completed, a de-identified version is stored in the repository 230 to support the queries of other patients. The de-identified clinical pathway versions can log the different clinical pathways that different patients may follow through a same guideline. That is, the de-identified clinical pathway versions can help identify deviations to a guideline that may occur under certain circumstances, and the reasons for such deviations.

Patients and/or medical care providers may indicate to the PCP Manager 246 the relationship between a PCP record 236, a published medical guideline 232, and/or a PHR 238. The PCP Manager 246 may also infer such relationships, possibly also indicating any ambiguity in the inference, e.g., by indicating that a PHR 238 corresponds to one or more of several currently active guidelines. According to some embodiments of the present disclosure, guideline(s) may not be reported along with the clinical pathway corresponding to the guideline(s). For example, N guidelines may be active, or potentially active, e.g., as being applicable to a particular patient, and the clinical pathway of the particular patient can be annotated with the active and/or potentially active guidelines as an aid to informing the patient and/or medical care provider regarding the possible medical process(es) to which the patient may be subjected.

The workflow engine 222 can also integrate with a Drools-expert rule engine, for example, which can be responsible for rule-based reasoning. Rules can be stored in a workflow engine-specific language, stored in memory, and executed by the Drools-expert, for example. When a decision node is reached by the workflow engine 222 in a medical process, for example, a decision routine can be executed in order to present available options to both patients and/or medical care providers. For example, the open source PHR tool MYOSCAR can be used to develop a patient-oriented portal.

A guideline, e.g., a medical guideline, can be formatted for computer implementation by defined relationships, such as where T is the set of all tasks, N is the set of all control nodes, e.g., decision nodes, action nodes, task nodes, end nodes, etc., and E is the set of all edges connecting task nodes and control nodes. A goal can be the objective of this guideline. The task node can denote various task types, and the control node can denote four basic control-flow constructs. Selecting a guideline for implementation at a particular medical care facility can require an agreement among care professionals at that medical care facility and its patients, since different kinds of guidelines can exist with different sources and goals. Conflicts can exist between various guidelines. Thus, in practice, guidelines can be adapted to a specific medical care facility and/or healthcare organization setting.

A clinical pathway implements medical guidelines after they are tailored to local and individual circumstances, e.g., availability of resources. In the clinical pathway, different tasks (performed by clinicians involved in the care) are defined and optimized in a logical time sequence. Outcomes are tied to specific interventions, e.g. taking medication for a week or an angioplasty procedure might reduce blood pressure. In general, a clinical pathway may be developed from many different guidelines. A clinical pathway can be the result of evidence-based medicine that drives the treatment of a specific group of patients with a predictable outcome.

A clinical pathway can be derived from a particular guideline, or from a plurality of guidelines. A particular guideline, and the adaptation thereof, can be made based on local settings. One example clinical pathway is represented in the clinical workflow 104 shown in FIG. 1.

A clinical pathway can associate two medical guidelines, e.g., from AHRQ, related to particular medical condition(s), e.g., heart failure management. For example, a first medical guideline can be a general one for evaluation and care of patients with heart failure, which might invoke a second medical guideline for initial evaluation. For detection and treatment tasks, other guidelines, e.g., medical and/or administrative, can be triggered depending on patient conditions and choices made by the attending medical care providers. In this way, the clinical pathway can guide the treatment of patients with heart failure in a structured, process-driven manner. When the clinical pathway is initialized for an actual case, human still take control.

In addition, the PCP can document the actual execution for a specific patient within a specific medical process and/or along a particular care episode. Thus, the PCP can be used to keep track of medical decisions, e.g., prescribe an ACE inhibitor or Beta blocker, actions, e.g., dosage for ACE inhibitor medication, and patient outcomes, e.g., normal body temperature, in chronological order for each patient situation. Deviations from guidelines can be recognized during the actual execution so that unexpected decisions can be explained and justified by the medical care providers and/or patient. A final outcome, e.g., the patient is cured, or ultimately passes away, indicates the end of a PCP. The PCP can be a result of clinical decision making.

A PCP is the execution log of a clinical pathway, such as where there is a decision or action taken, and can indicate a patient outcome or state. Each task can be associated with a time t to denote the time of occurrence. The PCP can capture other elements such as assessment and variance.

With respect to patient-centric decision making, medical knowledge for decision points is formulated in rules to derive recommendations based on best practice or patient choices. Patient preferences are collected through context-building with a PCM and giving patients more responsibility.

FIG. 3 illustrates a decision tree of a partial Patient Conversation Model (PCM) in accordance with one or more examples of the present disclosure. A medical decision can be context-dependent. Context can be patient specific. Context can be collected and summarized by asking questions of a patient at a previous point of time to the point in time at which the context is used. For example, context used during detection (110 shown in FIG. 1) and treatment (111 shown in FIG. 1) can be collected prior to that point in a clinical workflow (104 shown in FIG. 1), such as during admission (109 shown in FIG. 1), or during a preceding medical process and/or care episode.

FIG. 3 shows the partial PCM represented in a decision tree. The decision tree can include a number of decision nodes 364 corresponding to questions, and branches, e.g., 366 and 368, corresponding to answers associated with the question. A medical guidelines portion 332 can be derived from medical guidelines or rules. A practical guidelines portion 360 can be developed based on practical experience, and a patient needs and preferences portion 362 can be developed based on patient needs.

The medical guidelines portion 332, the practical guidelines portion 360, and patient needs and preferences portion 362 can be presented to the patient and/or medical care provider as questions 339, for example, such as those comprising the conversation model 250 shown in FIG. 2. The medical guidelines portion 332 and the practical guidelines portion 360 can be used to ascertain information relevant to the PHR 338, and the patient needs and preferences portion 362 can be to ascertain information relevant to the PPP 340.

A sequence of questions and answers may be related to a set of likely outcomes and issues that may affect patient experience. For example, a certain set of answers within the context of the guidelines may suggest that a certain diagnosis is likely and that a patient may need some additional helpful information that is not usually provided by a medical care provider. The set of questions may be reduced in quantity and/or scope using information from a patient's PHR. For example, some issues may only affect people of less than a certain age. The remaining outcomes and issues can be associated with medical processes that are presented to the patient to solicit and/or provide additional information that improves patient experience.

The relationships between guidelines can also be important. In some cases multiple guidelines may concurrently apply to a patient, e.g. say somebody who is a diabetic and also has respiratory dysfunction. The relationships between guidelines can be used to ensure that the two separate guidelines do not conflict with each other and any inconsistencies among them are resolved. Moreover, a patient being discharged from a medical care facility can transition from one guideline that is under the administration of the medical care facility to another guideline that may be governed by a social care agency. The PII may record a patient's steps as they receive services from each provider within a same medical process, the context model can utilize context information about the relationships between various guidelines so that a patient's experience can be modified, e.g., improved, based on the context of an applicable guideline when care is transferred, or shared, across multiple administrative domains, e.g., healthcare organizations. The patient can be made aware of an entity that is responsible for particular constraints and/or criteria for receiving services and/or the guideline(s) applicable to a particular medical care decision, treatment options, and/or available medical care providers. This can improve the patient's situational awareness of the healthcare system and/or particular healthcare organizations.

The example tree of questions illustrated in FIG. 3 can be made available for patients to answer at a time prior to detection 110 and treatment 111 in the clinical workflow 104 shown in FIG. 1. For example, a patient can enter her answers at home or while waiting for an examination, e.g., prior to admission 109 in FIG. 1, or during admission 109 shown in FIG. 1. As a result, the answers can be stored in the PIM, and subsequently used as context information. Some answers can become a part of the PHR, and some part of the PPP.

The conversation model gives the patient an opportunity to inquire of the provider. For example, if the PII determines a patient as possibly having hypertensive heart failure, a patient may interact with the PII to ascertain the treatment options and details associated with each option, e.g., expected recovery time, cost, side effects, and success rate. In general, when patients take the steps to proactively seek information regarding their illness, they are in a more advantageous position to share their specific knowledge and concerns with their medical care providers. Thus medical care providers can spend less time to make patients aware of readily accessible information while ensuring a patient-centric decision. In practice, other aspects of context can be considered, such as those related to clinical staff, e.g., expertise level, resources, availability, etc.

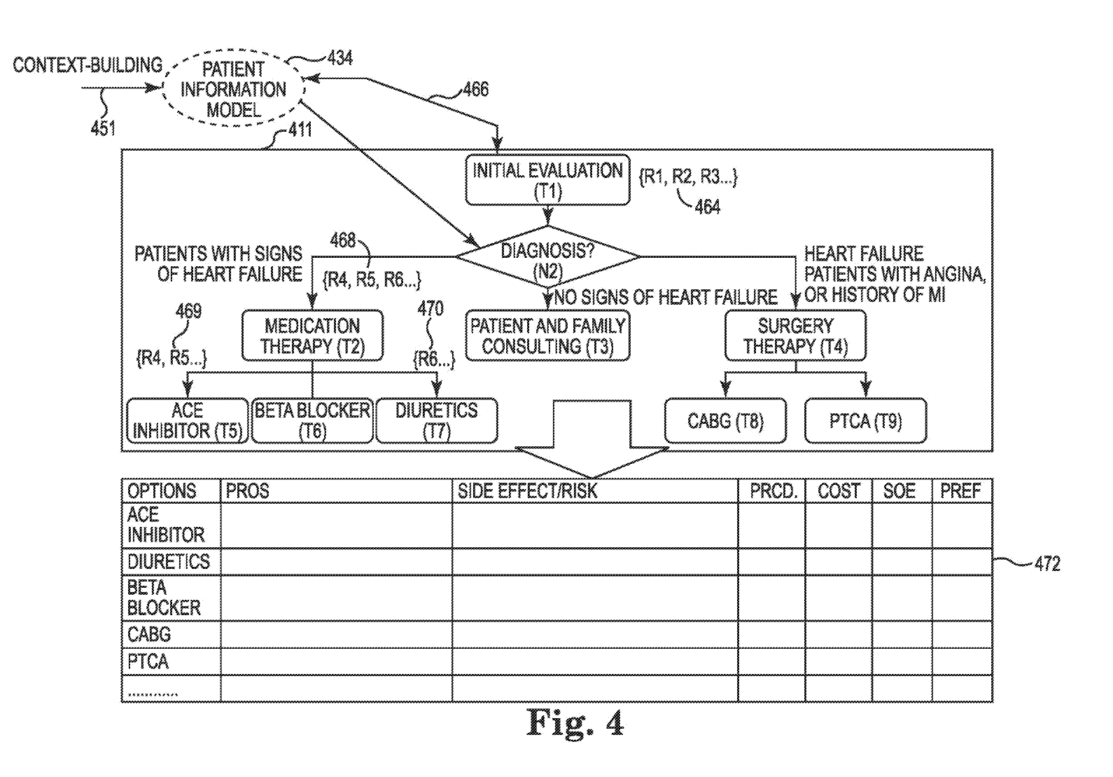

FIG. 4 illustrates a flow diagram of operation for a workflow engine portion of a computing system for implementing a PII in accordance with one or more examples of the present disclosure. FIG. 4 shows an example of workflow engine-implemented decision making at decision node N2 regarding determination of a medical diagnosis. As shown in FIG. 4, attributes of a PIM 434 include context information derived from context building activities 451.

Context-building is the process of obtaining patient information by conversing with patients, e.g., when they are waiting in a clinic, in a structured way. Many types of context information can also supported using the present approach. Thus, the decision algorithm integrates guidelines, patient needs and preferences, and suggests options. Finally, an action is determined based on the discussion and agreement between the patient and the medical care providers. All medical decisions, actions, and outcomes for each patient encounter are documented in a PCP which is a part of PIM.

The PII can, for example, detect any deviations from medical guidelines and request the medical care provider to enter reasons for any major deviations, which will be logged automatically, and remind the medical care provider perhaps at a later time. The PII helps to guide patient communication by semi-structured process models and coordinates activities among various participants. It helps to reduce the work required by the patient to interact with the system, e.g., duplicate data solicitation, and enables the patient's participation in various tasks by letting them know what to expect and what actions to take at all points throughout their care.

During an initial evaluation at T1, a patient might undergo a physical exam and/or diagnostic testing. Various results of the physical exam and/or diagnostic testing, e.g., high blood pressure, might be consistent with heart failure. The patient's PHR may include previous symptoms such as shortness of breath upon exertion and while sleeping, volume overload, and a little chest pain. Based on medical guidelines for heart failure, a decision tree might be invoked that includes a number of possible treatment options and a rule set 464.

The example rules acquired from guidelines for the example illustrated in FIG. 4 can be:
R1: If a patient has the following symptoms: awakening from sleep with shortness of breath or shortness of breath upon lying down or new-onset dyspnea on exertion, then the patient should undergo evaluation for heart failure unless PHR and physical exam clearly indicates a noncardiac cause [SOE=B].

R2: If a patient shows symptoms that are highly suggestive of heart failure Then the patient should undergo ECHO or RVG test to measure EF Even if physical signs of heart failure are absent [SOE=C].

R3: If a patient is suspected of clinically evident heart failure Then practitioners should perform: a chest x-ray; ECG; complete blood count; serum electrolytes . . . liver function tests; and urinalysis; If the patient is over 65 with no obvious etiology Then a T4 and TSH level should be checked [SOE=C].

R4: If a patient's systolic blood pressures <90 mmHg and there is a higher risk of complications Then prescribe ACE inhibitors managed by an experienced physician [SOE=A]

R5: If a heart failure patient has symptoms: fatigue or mild Dyspnea on exertion; and he has no other signs or symptoms of volume overload Then ACE inhibitors may be considered [SOE=C].

R6: If a patient has heart failure and signs of significant volume overload Then the patient should be started immediately on a diuretic [SOE=C].

Rules, such as medical rules can embody medical knowledge and are used to help make complex decisions in clinical pathways through logical reasoning. For example in FIG. 4, N2 is a decision node to decide the next step (treatment or further evaluation) based on patient diagnosis results. A number of medical rules can be associated with decision node N2. Integrating these rules and applying results from rule-based reasoning into a clinical pathway can implement evidence-based practice.

In addition, each rule can be associated with a strength of evidence (SOE) value to indicate its reliability. The SOE value can be obtained from an existing guideline, or other source, and may be adjusted, e.g., based on context information. For example, a three-level quality-rating system can be used for classifying SOE into levels A, B, and C. Rules from all categories can be automatically triggered, e.g., based on guidelines and PHR, and provide results to patients and medical care providers.

Reliability of a rule can be indicated by the SOE value in square brackets. The rules are categorized in a way that each rule is associated with the decision node in the clinical pathway illustrated in FIG. 4, and the various treatment options shown herein. For example, rule R1 is used at the initial examination (and diagnostic testing) (T1) or not (proceed to an End node). Similarly, rules R2 and R3 can also be tested at T1. Rules R2 and R3 can refer to another, e.g., different, medical guideline (not expanded in FIG. 4). The medication rules R4-R6 show which medication therapy is appropriate along with their SOE levels.

Treatment options might include a list of options, e.g., optionlist={T3 (patient and family counseling), T5 (ACE inhibitor), T6 (beta blocker), 17 (diuretics), T8 (CABG), T9 (PTCA)}. Treatment options may be categorized further into several intermediate classifications, such as medication therapy (T2) including T5, T6, and T7, and surgery therapy (T4) including T8 and T9.

The physical exam and/or diagnostic testing observations can be evaluated according to the rule set to determine appropriateness of the respective treatment options. For example, rule set 468, including rules R4, R5, R6, etc., applied to the PHR (including the physical exam and/or diagnostic testing observations) might define patients with signs of heart failure and suggest medication therapy (T2). More specifically, rule set 469, including rules R4, R5, etc., might indicate treatment with one class of drugs, e.g., T5, and rule set 470, including rules R6, etc., might indicate treatment with another class of drugs, e.g., T7. That is, the result of rule-based reasoning against the PHR can, for example, compute a result list result_list={T5, T7}, where only two rules are triggered for a PHR having specific information: R4 and R6. Other rules of rule set 464 might cause the result of rule-based reasoning against the PHR to conclude no signs of heart failure (thus suggesting T3) or heart failure patients with angina or history of MI (thus suggesting T4).

The process flow can proceed through each option in option_list. If a treatment option is triggered by a rule, e.g., R4, R5, it can be output to an interaction, e.g., with patient and/or medical care provider, along with an associated SOE value, and/or patient preference value. Otherwise, if a rule is not triggered, the value for its SOE can be “N/A”, indicating it is not applied. An output of the medical diagnosis decision tree can be summarized in a table 472, for example, where the advantages, side effects/risk, procedure, cost, SOE, and patient preference can be tabulated for presentation to the patient and/or medical care providers.

For this above-described example, although an ACE inhibitor and diuretics may be most highly recommended based on best practices, e.g., SOE, a patient and/or medical care provider can choose other option(s) in lieu of or in addition to the most highly recommended treatment options. For example, if the actual action taken is a Beta-blocker therapy, then the PII can be so informed, can detect such deviation in real-time, and ask the medical care provider to enter reasons for it (concurrently or perhaps at a later time).

Referring again to FIG. 2, the workflow engine 222 can be configured to implement medical decision making in support of the PII. Medical decision making can follow best practice through medical guidelines comprising various rules, and take into account patient-specific information from context-building. Medical guidelines can include decision nodes and associated branches. When a decision node D is reached, the rule set RS associated with D can be retrieved and run against a PHR to obtain results. Other options not triggered by rules can still be presented to patients and/or medical care providers with notation SOE=“N/A” as an indication that no guideline supports a particular option. In addition, SOE values and patient preference levels (from the PPP) can be shown. Patient-oriented decision making can be also reflected when decision node D is reached. An input to resolve the decision can be obtained from the PHR, the PPP, or further interaction with the patient and/or medical care provider. The output can be a list of options.

An example computer implementation of the above described medical decision making can be summarized in the following quasi-code:

```
      1. Put all the action nodes from the outgoing branches of D
into a list option_list
      2. Define rule set RS = rules associated with decision node D
      3. Run RS against PHR and get the result vector result_list //a
subset of option_list
      4. Define a temporary tuple <option, rule, pref>
      5. FOR each item op in option_list
      6. tuple.option = op.name //assign the option name
      7. tuple.pref = preference_table.findPref(op.name) //assign
preference from PPP
      8. If op is in result_list //this option is a result rule-based
reasoning or best practice
      9. tuple.rule = the content of the rule that trigger this action op
(with SOE value)
      10. Else //op is not option from guideline, no rule is applied but
this is still an option
      11. tuple.rule=“N/A”
      12. Insert tuple into options
      13. END
      14. Rank options first based on SOE (A-C), then based on
patient preference (high-low)
      15. Return options
```

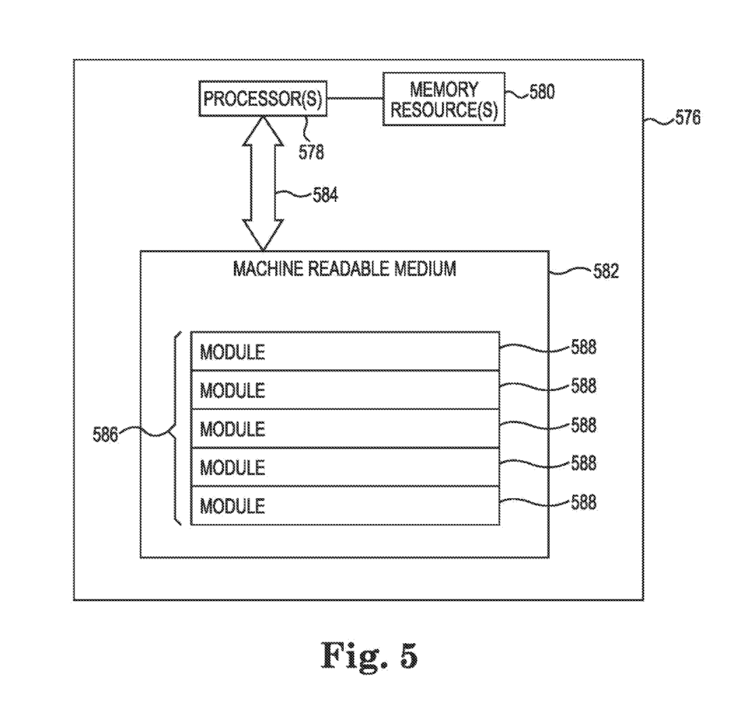

FIG. 5 illustrates a block diagram of an example machine readable medium in communication with processing resources in accordance with one or more examples of the present disclosure. A computing resource 576 can include processing resources 578 communicatively coupled to a machine readable medium (MRM) 582 and/or memory resources 580. The MRM 582 can be communicatively coupled with the processing resources 578 via a communication path 584. As used herein, processing resources 578 can include at least one processor, which can be arranged in a parallel processing arrangement. The MCM can be a tangible non-transitory computer readable medium 795 storing a set of computer readable instructions 586, e.g., software, for implementing a patient information interface, as described herein. The machine readable medium can be configured include various modules 588, for example.

A computing system, such as that shown in FIG. 2, can be comprised of a number of computing resources 576 communicatively coupled to a network. For example, a first computing device can have an associated data source, and may have one or more input/output devices, e.g., keyboard, electronic display. A second computing device can be communicatively coupled to the network, such that executable instructions may be communicated through the network between the first and second computing devices.

The first and/or second computing device may be further communicatively coupled to a production device, e.g., electronic display, printer, etc., and/or can also be communicatively coupled to an external computer-readable memory. The first and/or second computing device can cause an output to the production device, for example, as a result of executing instructions of one or more programs stored on non-transitory computer-readable medium, by the at least one processor, to implement a PII according to the present disclosure. Causing an output can include, but is not limited to, displaying text and images to an electronic display and/or printing text and images to a tangible medium, e.g., paper. Executable instructions to implement a PII may be executed by the first computing device and/or second computing device, stored in a database such as may be maintained in external computer-readable memory, output to production device, and/or printed to a tangible medium.

One or more additional computers may also be communicatively coupled to the network via a communication link that includes a wired and/or wireless portion. The computing system can be comprised of additional multiple interconnected computing devices, such as server devices and/or clients. Each computing device can include control circuitry such as a processor, a state machine, application specific integrated circuit (ASIC), controller, and/or similar machine.

Control circuitry of a computing resource 576 can have a structure that provides a given functionality, and/or execute computer-readable instructions that are stored on a non-transitory machine readable medium 582. The non-transitory machine readable medium 582 can be integral (as shown in FIG. 5), or communicatively coupled to the respective computing resource 576 in either a wired or wireless manner. For example, the non-transitory machine readable medium 582 can be an internal memory, a portable memory, a portable disk, or a memory located internal to another computing resource, e.g., enabling the computer-readable instructions to be downloaded over the Internet. The non-transitory machine readable medium 582 can have computer-readable instructions stored thereon that are executed by the control circuitry and/or the processing resources 578 to provide a particular functionality.

The non-transitory machine readable medium 582, as used herein, can include volatile and/or non-volatile memory. Volatile memory can include memory that depends upon power to store information, such as various types of dynamic random access memory (DRAM), among others. Non-volatile memory can include memory that does not depend upon power to store information. Examples of non-volatile memory can include solid state media such as flash memory, EEPROM, phase change random access memory (PCRAM), among others. The non-transitory computer-readable medium can include optical discs, digital video discs (DVD), Blu-ray discs, compact discs (CD), laser discs, and magnetic media such as tape drives, floppy discs, and hard drives, solid state media such as flash memory, EEPROM, phase change random access memory (PCRAM), as well as other types of machine-readable media.

A number of computing resources 576 can be used to implement the method(s) of the present disclosure, in whole or part. The PII can be implemented using appropriately configured hardware and/or machine readable instructions. Various portions of the PII may be discretely implemented and/or implemented in a common arrangement.

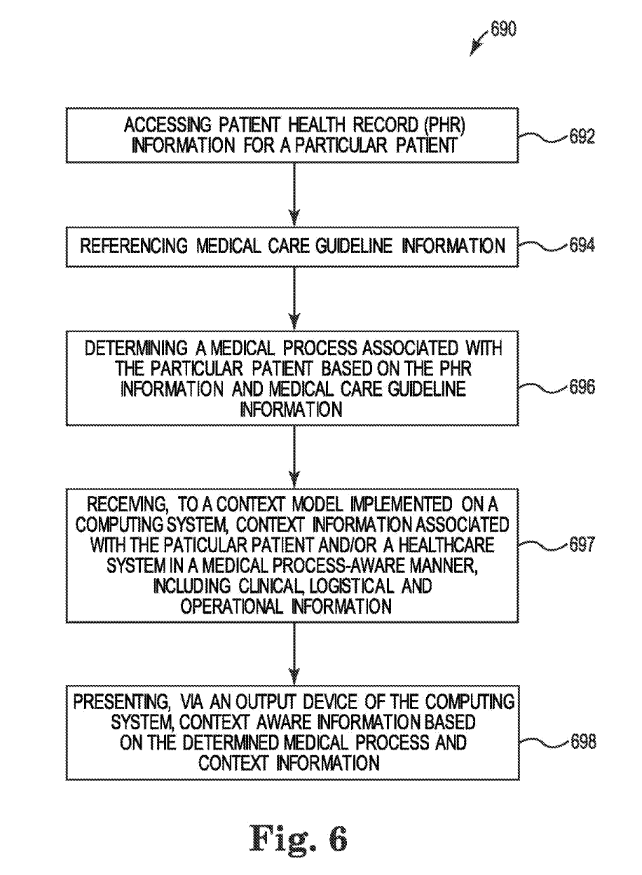

FIG. 6 illustrates a method 690 for PII in accordance with one or more examples of the present disclosure. The method for PII 690 can include accessing PHR information for a particular patient, as shown at 692, and referencing medical care guideline information, as shown at 694. As indicated at 696, method 690 further includes determining a medical process associated with the particular patient based on the PHR information and medical care guideline information, and at 697 context information associated with the particular patient and/or a healthcare system is received in a medical process-aware manner to a context model implemented on a computing system, including clinical, logistical and operational information. Context aware information is presented, via an output device of the computing system based on the determined medical process and context information, as shown at 698.

Although an example order is shown for the method 690 illustrated in FIG. 6 as indicated by the arrows, according to some embodiments of the present disclosure, the method can be implemented using a different arrangement of actions than that shown in FIG. 6. For example, referencing medical care guideline information 694 may occur prior to accessing PHR information for a particular patient 692.

Some embodiments of the present disclosure help to educate patients to improve the quality and efficiency of interactions with care providers. Disclosure via the PII can anticipate information that will be needed from patients so it can be gathered in advance, and educate patients about possible outcomes so that they can have improved discussions with providers. The PII can have knowledge of the relationship between various guidelines, e.g., medical, administrative, and the hand-off of care between medical care providers. This context information can be used to educate a patient, and thereby improve the patient's situational awareness of a healthcare system even when no provider is accountable for the hand-off. The PII disclosed by the present disclosure can anticipate problems and issues that are common to patients in similar situations and are able to provide information without the patient having to interrupt medical care providers in their work. The PII can help to improve patient safety, satisfaction, and outcome while improving the efficiency of the healthcare system.

The formal process-driven framework of the PII can streamline patient-centric care and improve patient-provider communication. As such, the PII can also lead to patients having better access health services and taking more responsibility in their health management, as well as reducing the burden on healthcare professionals while enabling greater efficiency, improved safety and higher quality.

The PII of the present invention augments a patient's PHR with contextual information that includes clinical information such as a patient's care process and care preferences. The context information is input to a context model. The context information can include information about the processes in a healthcare system, the relationship between processes in a healthcare system, and the protocols that should be followed by a patient to obtain a service from a medical care provider. Context information and the context model can be used to guide interactions with the patient to improve both the patient's situational awareness of the healthcare system and the patient's experience including safety, satisfaction, and outcome.

The PII can mitigate patient questioning of medical care provider staff and/or a need for the patient to have to search for information in some other fashion. Because the patient does not know what they do not know they may be unaware of information that the PII provides to improve safety, satisfaction, and outcome. Additionally, medical care provider staff may not always be aware of such information, or up to date information, that is relevant to a patient or have time to communicate it, or be able to communicate it in an appropriate language. Some services that a patient is entitled, or able, to receive may not be revealed to a patient, or the protocol for properly obtaining those services may not be revealed. In some cases providers may be responsible for their own interactions with a patient but are not knowledgeable or perhaps even accountable for the hand-off of a patient to another provider. Logistical tasks are simplified for patients by providing context aware information and instructions. By timely presenting such context aware information and instructions to the patient, the PII can increase the likelihood that a patient uses intended services at the appropriate time in an informed manner, thereby improving outcomes and making more effective use of the healthcare system.

The above specification, examples and data provide a description of the method and applications, and use of the system and method of the present disclosure. Since many examples can be made without departing from the spirit and scope of the system and method of the present disclosure, this specification merely sets forth some of the many possible example configurations and implementations.

Although specific examples have been illustrated and described herein, those of ordinary skill in the art will appreciate that an arrangement calculated to achieve the same results can be substituted for the specific examples shown. This disclosure is intended to cover adaptations or variations of one or more examples provided herein. The above description has been made in an illustrative fashion, and not a restrictive one. Combination of the above examples, and other examples not specifically described herein will be apparent upon reviewing the above description. Therefore, the scope of one or more examples of the present disclosure should be determined based on the appended claims, along with the full range of equivalents that are entitled.

Throughout the specification and claims, the meanings identified below do not necessarily limit the terms, but merely provide illustrative examples for the terms. The meaning of “a,” “an,” and “the” includes plural reference, and the meaning of “in” includes “in” and “on.” “Example,” as used herein, does not necessarily refer to the same example, although it may.

As used herein, the term “includes” means includes but not limited to, the term “including” means including but not limited to. The term “based on” means based at least in part on. The term “document,” as used herein, includes but not limited to, electronic files such as web pages and word processing files, among others.

In the foregoing discussion of the present disclosure, reference is made to the accompanying drawings that form a part hereof, and in which is shown by way of illustration how examples of the disclosure may be practiced. These examples are described in sufficient detail to enable those of ordinary skill in the art to practice the examples of this disclosure, and it is to be understood that other examples may be utilized and that process, electrical, and/or structural changes may be made without departing from the scope of this disclosure.

Some features are grouped together in a single example for the purpose of streamlining the disclosure. This method of disclosure is not to be interpreted as reflecting an intention that the disclosed examples of the present disclosure have to use more features than are expressly recited in each claim. Rather, as the following claims reflect, inventive subject matter lies in less than all features of a single disclosed example. The following claims are hereby incorporated into the Detailed Description, with each claim standing on its own.

What is claimed:

1. A method for providing a patient information interface, comprising:
accessing patient health record (PHR) information for a particular patient;
referencing medical care guideline information;
determining a medical process associated with the particular patient based on the PHR information and medical care guideline information;
receiving, to a context model implemented on a computing system, context information associated with the particular patient and/or a healthcare system in a medical process-aware manner, including clinical, logistical and operational information; and
presenting, via an output device of the computing system, context aware information based on the determined medical process and context information.

2. The method of claim 1, wherein receiving clinical information associated with the healthcare system includes:
identifying one or more medical care providers available to the particular patient;
recording those of the one or more medical care providers being used by the particular patient; and
storing medical care preference information associated with the particular patient.

3. The method of claim 1, wherein:
receiving logistical information associated with the healthcare system includes ascertaining a current physical location of the particular patient, and determining proximity of the current physical location of the particular patient to a respective location of one or more identified medical care providers that can provide a medical care service that corresponds to the determined medical process; and
wherein presenting context aware information to the particular patient includes providing instructions to receive a location-aware medical care service.

4. The method of claim 1, wherein presenting context aware information to the particular patient includes:
presenting, via the output device of the computing system, context aware information to the particular patient and/or medical care provider; and
providing instructions on how to receive a medical care service.

5. The method of claim 1, further comprising implementing interactions with the particular patient to obtain and provide information related to medical care guideline information including:
one or more medical care treatment options available to the particular patient based on the determined medical process; and
one or more likely outcomes associated with each of the one or more medical care treatments options and medical variables that are typical for similarly-situated patients.

6. The method of claim 5, wherein presenting context aware information to the particular patient includes reporting to the particular patient those of the one or more medical care treatment options available to the particular patient that is best aligned with medical care preference information associated with the particular patient, along with a reason supporting each best-aligned medical care treatment option.

7. The method of claim 5, wherein presenting context aware information to the particular patient includes reporting to the particular patient those of the one or more medical care treatment options available to the particular patient that is most likely to be recommended by the a medical care provider, along with reasoning therefor.

8. The method of claim 1, further comprising presenting, via the output device of the computing system to a care provider of the particular patient, one or more medical care treatment options recommended by the medical care guideline information based on the determined medical process and context information.

9. The method of claim 1, wherein receiving context information associated with the particular patient and/or the healthcare system includes storing the received context information in a repository, and wherein receiving operational information associated with the healthcare system includes defining a relationship between medical care guideline information based on a policy of the healthcare system.

10. The method of claim 1, further comprising:
recording progress of the particular patient through the determined medical process including a sequence of interactions between the particular patient and one or more medical care providers, wherein presenting context aware information to the particular patient includes providing a process-oriented view of records organized according to a medical care episode.

11. The method of claim 1, wherein obtaining medical care guideline information includes selecting among answers to a question of the medical care guideline information applied to the particular patient, the question corresponding to a decision node of a decision tree and the answers corresponding to branches of the decision tree.

12. A non-transitory computer-readable medium storing a set of instructions executable by a processing resource to cause a computer to:
access patient health record (PHR) information for a particular patient;
reference medical care guideline information;
determine a medical process associated with the particular patient based on the PHR information and medical care guideline information;
receive, to a context model implemented on a computing system, context information associated with a healthcare system, including clinical, logistical and operational information; and
present, via an output device of the computing system, context aware information to the particular patient based on the determined medical process and context information.

13. The medium of claim 12, further comprising the non-transitory computer-readable medium storing instructions executable by the processing resource to cause the computer to receive logistical information associated with the healthcare system includes ascertaining a current physical location of the particular patient, and determining proximity of the current physical location of the particular patient to a respective location of one or more identified medical care providers that can provide a medical care service that corresponds to the determined medical process; and
wherein the context aware information presented to the particular patient includes providing instructions to receive a location-aware medical care service.

14. A patient information interface, comprising:
a processing resource in communication with a non-transitory computer readable medium, wherein the non-transitory computer readable medium includes a set of instructions and wherein the processing resource executes the set of instructions to:
access patient health record (PHR) information for a particular patient;
reference medical care guideline information;
determine a medical process associated with the particular patient based on the PHR information and medical care guideline information;
receive, to a context model implemented on a computing system, context information associated with a healthcare system, including clinical, logistical and operational information; and
present, via an output device of the computing system, context aware information to the particular patient and a medical care provider based on the determined medical process and context information.

15. The patient information interface of claim 14, wherein the processing resource executes the set of instructions to receive logistical information associated with the healthcare system includes ascertaining information according to insurance coverage information of the particular patient, and determining availability of a medical care service that corresponds to the determined medical process based on the insurance coverage information of the particular patient; and
wherein the context aware information presented to the particular patient includes providing instructions regarding medical care service availability.
